(19) United States
(12) Patent Application Publication
(10) Pub. No.: US 2005/0287524 A1
(43) Pub. Date: Dec. 29, 2005

(54) Differential serological diagnosis of equine infectious anemia virus infected and vaccinated horses using recombinant S2 protein

(76) Inventors: Ronald C. Montelaro, Wexford, PA (US); Charles J. Issel, Campton, KY (US); Sha Jin, Pittsburgh, PA (US)

Correspondence Address: TRASK BRITT, P.O. BOX 2550, SALT LAKE CITY, UT, 84110, US

(21) Appl. No.: 11/210,358

(22) Filed: Aug. 24, 2005

Related U.S. Application Data
(63) Continuation-in-part of application No. 10/409,397, filed on Apr. 7, 2003.
Application No. 10/409,397 is a continuation of application No. 10/369,792, filed on Feb. 19, 2003.
Application No. 10/369,792 is a division of application No. 09/658,547, filed on Sep. 9, 2000, now Pat. No. 6,585,978.

(51) Int. Cl. C12Q 1/70

(52) U.S. Cl. 435/5

(57) ABSTRACT

We describe here the development and optimization of an embodiment of a new serologic EIAV diagnostic ELISA assay to detect serum antibodies to the EIAV S2 protein that are produced in infected horses, but not in horses inoculated with the EIAV_{UK}ΔS2 vaccine virus. An embodiment of the test S2 protein antigen was developed using the S2 gene sequence from the EIAV_{UK} strain of virus and a series of modifications to facilitate production and purification of the diagnostic antigen, designated HS2G. Using this embodiment of an HS2G as antigen, we describe the development of an affinity ELISA (NN-ELISA) that provides a sensitive and specific detection of S2-specific serum antibodies in experimentally and field infected horses (22/24), without detectable reactivity with immune serum from uninfected (12/12) or vaccinated (29/29) horses.

### RELATED APPLICATION

This application is a continuation in part of U.S. Ser. No. 10/409,397, filed Apr. 7, 2003, which is a continuation of Ser. No. 10/369,792, filed Feb. 19, 2003, which is a divisional of Ser. No. 9/658,547, filed Sep. 9, 2000, now U.S. Pat. No. 6,585,978, the texts of which are all hereby incorporated by reference, and, this application claims priority to provisional 60/604,305, filed Aug. 24, 2004, the text of which is hereby incorporated by reference.

### FIELD OF THE INVENTION

Embodiments of the present invention generally relate to a new serologic equine infectious anemia virus (EIAV) diagnostic assay to differentiate an inoculated equine from an equine infected with EIAV.

### BACKGROUND OF THE INVENTION

The macrophage-tropic animal lentivirus equine infectious anemia virus (EIAV) causes a persistent infection in horses and has been recognized as an important veterinary infectious disease for over 150 years (12, 22, 28). EIAV, transmitted by blood-feeding insects and fomites such as contaminated needles, is characterized by recurring cycles of viremia and clinical episodes that include fever, anemia, thrombocytopenia, edema, and wasting. Following 6-12 months of this chronic disease stage, most animals progress to an inapparent stage absent overt clinical disease (8, 12, 22, 28). Because of the economic importance of controlling EIAV infections, combined with the successful use of EIAV as an animal lentivirus model for HIV-1, the development of an effective vaccine to EIAV has been aggressively pursued (7, 15, 20, 24-25, 33).

EIAV infection of horses is a global veterinary concern and a disease for which there is currently no vaccine. Therefore, control of this viral infection is achieved in certain countries by the destruction of EIAV-infected horses identified by serological diagnostic assays that detect antibodies to viral core or envelope proteins. The development of an effective vaccine to lentiviruses in general and EIAV in particular has been inhibited by a confounding array of immune evasion and persistence mechanisms intrinsic to these viruses, as frequently highlighted by AIDS vaccine efforts. The implementation of an effective EIAV vaccine, however, is even further complicated by current regulatory policies in certain countries that will require an ability to distinguish infected from vaccinated horses. We have previously described an experimental live attenuated EIAV vaccine, EIAV_{UK}ΔS2, that appears to achieve a high level of protection from exposure to virulent virus challenge (20). Thus, the goal of the current study was to develop a companion serological diagnostic assay that is seropositive for infected horses while being seronegative for uninfected and vaccinated horses. The data presented here indicate that a diagnostic assay based on detecting serum antibody reactivity to EIAV S2 antigen has the required specificity and sensitivity for further development for application in detecting and distinguishing EIAV-infected horses from vaccinated horses using a practical and simple ELISA format that can be optimized for high throughput.

The genetic organization of EIAV, as with HIV, SIV and FIV contains only three accessory genes (S1, S2 and S3), in addition to the gag, pol and env genes common to all retroviruses. The S1 open reading frame (ORF) encodes the viral Tat protein, a transcription trans activator that acts on the viral long-terminal-repeat (LTR) promoter element to stimulate expression of all viral genes. The 53 ORF encodes a Rev protein, a post-transcriptional activator that acts by interacting with its target RNA sequence, named the Rev-responsive element (RRE), to regulate viral structural gene expression. The S2 gene is located in the pol-env intergenic region immediately following the second exon of Tat and overlapping the amino terminus of the Env protein (see FIGS. 1, 2a and 2b). It encodes a 65-amino-acid protein with a calculated molecular mass of 7.2 kDa, which is in good agreement with the size of an in vitro translation product. S2 appears to be synthesized in the late phase of the viral replication cycle by ribosomal leaky scanning of a tricistronic mRNA encidong Tat, 52 protein, and Env, respectively. The ORF coding for the S2 protein of EIAV is highly conserved in all published EIAV sequences and contains three potential functional motifs (FIG. 2a). Antibodies to S2 protein can be found in sera from experimentally and naturally infected horses, indicating that S2 is expressed during EIAV replication in vivo. These observations suggest that S2 is likely to perform an important role in the virus life cycle. A discussion of the function of S2 is found in Li et al (J. Virol., October 1998, p 8344-8348), incorporated herein by reference.

This laboratory has previously described the development of a live-attenuated EIAV vaccine based on an engineered proviral construct with a mutated S2 gene, EIAV_{UK}ΔS2 vaccine (20). The EIAV S2 gene is located in the pol-env intergenic region, overlaps the amino terminus of the EIAV envelope proteins, and is synthesized in the late phase of the viral replication cycle (3, 5). While the biologic function of this 65 amino acid accessory protein is unknown, previous studies have indicated that S2 is not essential for EIAV replication in vitro (18, 27), but that the absence of S2 severely reduces EIAV replication and virulence in experimentally infected horses (19). In vivo studies of EIAV_{UK}ΔS2 as a potential attenuated viral vaccine demonstrated protection from infection in vaccinated horses subjected to low-dose multi-exposure intravenous virulent virus challenge (to mimic field exposure) or to a single high-dose i.v. virus challenge (20).

In light of these promising initial observations on the efficacy of a live attenuated EIAV vaccine, the EIAV_{UK}ΔS2 may represent an effective vaccine for the control of EIAV infections. Thus, the development of a diagnostic assay that can effectively identify and discriminate EIAV-infected animals from EIAV_{UK}ΔS2-vaccinated horses become a necessary advancement. In countries where veterinary regulatory policies are established, EIAV infections are currently controlled by detection and destruction or isolation of virus infected horses. Approved diagnostic assays for EIAV infection are based on detection of serum antibodies to the capsid protein p26, transmembrane glycoprotein gp45, and surface glycoprotein gp90 in various assay formats (1, 2, 9, 14, 17, 21, 29-32). At present, the United States Department of Agriculture (USDA) primary EIA testing systems include the agar gel immunodiffusion assay (AGID or Coggins test) and ELISA assays, all of which are based on the detection of serum antibodies to EIAV p26 or gp45 (4, 13). Attenuated proviral vaccines containing a mutated S2 accessory gene still express the full complement of EIAV core and envelope proteins. Therefore, horses inoculated with the EIAV_{UK}ΔS2 become seropositive in current diagnostic assays for EIAV infection, posing a complication to current regulatory procedures used to control infection and disease in horses.

To address this important regulatory issue, we sought to develop and evaluate the reliability of an EIAV S2-based serological assay in detecting and discriminating EIAV-infected horses from vaccinated horses inoculated with EIAV_{UK}ΔS2. We describe in the current report the engineering and production of a novel recombinant S2 protein antigen and the optimization of an S2 antigen-based ELISA for the detection of reactive antibodies in serum from experimentally and field infected horses, without reactivity to antibodies in immune serum from horses inoculated with the attenuated EIAV vaccine. These data demonstrate for the first time the potential of an S2-based serological diagnostic to accurately identify and discriminate horses infected with wild type EIAV from horses inoculated with S2-attenuated vaccine virus.

### SUMMARY OF THE INVENTION

Generally, embodiments of the present invention relate to new serological EIAV diagnostics for differentiating an equine inoculated with an equine infectious anemia virus (EIAV) vaccine and an EIAV infected equine. Various embodiments of the present invention comprise a serological assay for detecting antibodies to a gene of EIAV wherein the absence of antibodies to the accessory gene indicates an inoculated equine. In an embodiment, the gene is an accessory gene. In general, various embodiments of the present invention comprise diagnostics comprising an enzyme linked immunosorbant assay (ELISA), an immunodiffusion test, a fluorescent antibody test (FA), and/or any other test that can be used to detect antibodies in mammals. The diagnostics of the present invention comprise diagnostic methods and diagnostic kits.

Accordingly, various methods of the present invention generally comprise a diagnostic method for differentiating an equine inoculated with an equine infectious anemia vitus (EIAV) vaccine, wherein the vaccine comprises a gene mutated EIAV, and an EIAV infected equine comprising a serological assay for detecting antibodies to the gene. In various embodiments, the serological assay is a serological assay selected from the group consisting of an enzyme linked immunosorbant assay (ELISA), an immunodiffusion test, a fluorescent antibody test (FA), and any other test that can be used to detect antibodies in mammals. In further embodiments, the diagnostic is a PCR based diagnostic wherein the serological assay detects the presence or absence of a gene, gene sequence, or fragment thereof.

Further embodiments generally comprise a diagnostic kit for differentiating an equine inoculated with an equine infectious anemia virus (EIAV) vaccine, wherein the vaccine comprises a gene mutated EIAV, and an EIAV infected equine comprising a serological assay for detecting antibodies to the gene wherein the absence of antibodies to the gene indicates an inoculated equine.

Further embodiments generally comprise a diagnostic method for differentiating an equine inoculated with a gene mutated equine infectious anemia virus (EIAV) marker vaccine and an EIAV infected equine comprising a serological assay for detecting antibodies to the gene wherein the presence of antibodies to the gene indicates an inoculated equine.

### BRIEF DESCRIPTION OF THE FIGURES

FIG. 1. Table with various primers, plasmids, and promoters used for RT-PCR.

FIG. 2. Sequence alignment of the parental and codon-optimized and EIAV_{UK} S2 genes sequences. The parental S2 sequence is as reported in Cook et al (5). The codon-optimized sequence was derived using the Codon Optimization Database (www.kazusa.or.jp).

FIG. 3. Analysis of the expression level of S2 mRNA and protein in transfected COS7 cells. (A) COS7 cells were transfected with the individual expression plasmids outlined in Table 1. Cell lysate was prepared from 1.5×10⁵ cells harvested 48 hours post transfection, and mRNA was extracted and analyzed by RT-PCR using the primers indicated in Table 1. (B) Western blot analysis of S2 expression in COS7 cells. Approximately 3.2×10⁶ cells were harvested, lysed and immunoblotted using rabbit anti-S2 antibodies.

FIG. 4. The expression of HS2G in E. coli XL:p80LOS2GFP. (A) Flow cytometric analysis of the expression of HS2G in E. coli transformed with XL:p80LOS2GFP (bond lines). Wide type E. coli served as a control (Shaded). (B) Coomassie blue visualization of HS2G protein resolved by 4-20% SDS-PAGE. E. coli collected 4 h post induction were lysed under different conditions. Lane 1, cell lysate prepared under a native conditions; lane 2, cell lysate prepared under denaturing conditions; lane 3, HS2G purified under denaturing conditions; lane M, standard protein markers. P.i.: post induction. (C) Immunoblot of HS2G with rabbit anti-EIAV S2 antibody as primary antibody (1:200 dilution) and anti-rabbit IgG HRP as the secondary antibody (1:10,000 dilution). Lane 4, HS2G in the cell lysate prepared under native conditions; lane 5, HS2G in the cell lysate prepared under denaturing conditions; lane 6, HS2G purified under denaturing conditions.

FIG. 5. Evaluation of the reactivity of antigens to rabbit anti-S2 polyclonal antibodies and EIAV-infected horse serum in standard ELISA and NN-ELISA. HS2G reactivity to rabbit anti-S2 polyclonal antibodies (1:200 dilution) and to EIAV-infected horse immune serum (1:25 dilution) in the standard ELISA (A) or the affinity NN-ELISA (B) format.

FIG. 6. Comparison of HS2G reactivity to horse sera using the standard ELISA (Std-ELISA) and NN-ELISA (HS2G-NN) formats. Equal amounts of purified HS2G protein were analyzed in parallel in either the standard or affinity ELISA format against a panel of reference horse serum samples: “Negative”—uninfected horses shown to be seronegative in standard EIAV p26 diagnostic assays; “Positive”—virus-infected horses shown to be seropositive in standard EIAV p26 diagnostic assays; “Vaccinated”—horses experimentally inoculated with attenuated S2 vaccine strains of EIAV and seropositive in standard p26 diagnostic assays. All horse serum samples were used at a dilution of 1:25.

FIG. 7. Evaluation of HS2G NN-ELISA to identify serum samples from uninfected, infected, and vaccinated horses. The panel of reference serum samples included uninfected horses (1-7), horses persistently infected with EIAV (8-25), and horses vaccinated with attenuated S2 vaccine strains (26-46). All horse sera were used at a dilution of 1:25 in HS2G NN-ELISA. The affinity ELISA procedures for detecting serum antibodies to the HS2G antigen or the EIAV p26 protein were as described in Materials and Methods.

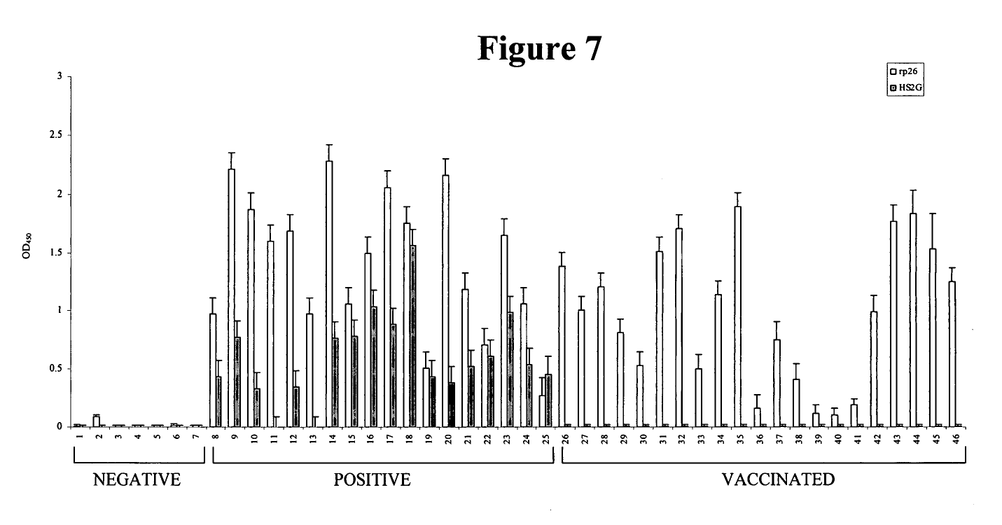

### DETAILED DESCRIPTION OF THE INVENTION

As used herein, the term “diagnostic” or “diagnostic assay” means and refers to a diagnostic method, a diagnostic test, or diagnostic test method.

As used herein, the term “gene amplification” means and refers to a process by which the cell increases the number of a particular gene within the genome, the process by which the number of copies of a chromosomal segment is increased in a cell.

As used herein, the term “equine” means and refers to an organism of the family equidae.

As used herein, the term “fragment” means and refers to a DNA or amino acid sequence comprising a subsequence of one of the nucleic acid sequences or polypeptides of the invention. Said fragment is or encodes a polypeptide having one or more immunoreactive and/or antigenic determinants of an EIAV related polypeptide, i.e. has one or more epitopes which are capable of eliciting an immune response in an equine and/or is capable of specifically binding to a complementary antibody. Methods for determining usable polypeptide fragments are outlined below. Fragments can inter alia be produced by enzymatic cleavage of precursor molecules, using restriction endonucleases for the DNA and proteases for the polypeptides. Other methods include chemical synthesis of the fragments or the expression of polypeptide fragments by DNA fragments.

As used herein, the term “nucleic acid sequence” means and refers to a polymeric form of nucleotides of any length, both to ribonucleic acid sequences and to deoxyribonucleic acid sequences. In principle, this term refers to the primary structure of the molecule. Thus, this term includes double and single stranded DNA, as well as double and single stranded RNA, and modifications thereof.

As used herein, the term “PCR method/process/procedure” and/or “PCR” and/or “PCR amplification” and/or “amplification” is a procedure capable of amplifying an intended DNA fragment as much as several hundred thousand-fold, or as little as may be needed, by repeating a process comprising the steps of dissociation of a DNA strand into single strands, binding of primers with at least one single strand, sandwiching a particular region of the DNA strand, and a DNA synthesis reaction by the action of a DNA polymerase.

As used herein, the term “polypeptide” means and refers to a molecular chain of amino acids with a biological activity, does not refer to a specific length of the product and if required can be modified in vivo or in vitro, for example by glycosylation, amidation, carboxylation or phosphorylation; thus inter alia, peptides, oligopeptides and proteins are included.

Generally, various embodiments of the present invention relate to a diagnostic method for differentiating an equine inoculated with an equine infectious anemia virus (EIAV) vaccine and an EIAV infected equine. Various embodiments of the diagnostics of the present invention can differentiate equines vaccinated with vaccines comprising a gene mutated EIAV. In various embodiments, mutations in an EIAV may comprise a mutation selected from a mutation in one of the three accessory genes (S1, S2 and S3), the gag gene, the pol gene, the env gene, virion proteins, including, but not limited to the capsid protein p26, transmembrane glycoprotein gp45, and/or surface glycoprotein gp90, among others.

Further vaccines for which diagnostic methods of the present invention are effective comprise marker vaccines for equines. General embodiments of marker vaccines for an EIAV vaccine comprise a mutation, such as, but not limited to, a deletion, in the capsid protein p26, transmembrane glycoprotein gp45, and/or surface glycoprotein gp90. However, other mutations suitable for marker vaccines would be apparent to one of ordinary skill in the art. Marker vaccines generally further comprise an insertion and/or substitution of another protein, such as a foreign protein in the location of the deleted protein. In an embodiment of such a vaccine, a p30 protein of a Visna Virus is substituted for at least one of the p26, the gp45, or the gp90 protein. However, other suitable foreign proteins may be used for substitution. Diagnostics of the present invention may then detect the presence of absence of the foreign protein. The presence of the foreign protein or gene would indicate a vaccinated equine.

In an embodiment of a vaccine with a deletion of the p26 protein and insertion of the p30 protein, a serological assay could detect the presence of antibodies to the p26 or p30 proteins. If p26 antibodies were present in horses that were tested it would indicate that the horse had been infected with EIAV. Horses that had been vaccinated with a gene-mutated EIAV construct containing a non-functional p26 gene would not contain p26 antibodies in their serum. Horses that had been vaccinated with a gene-mutated EIAV construct containing a p30 gene insertion would contain p30 antibodies in their serum. Thus, vaccinated horses could be differentiated from infected horses. The PCR-based assays would be used to detect the presence or absence of gene sequences within the horse. For instance, if a horse had been infected with a wild-type EIAV, it would contain the gene sequence for wild-type p26. However, equines immunized with vaccines comprising a gene-mutated EIAV, particularly one wherein the p26 gene comprised deletions or specific mutations would not contain the gene sequence for wild-type p26. Alternatively, horses that had been vaccinated with a gene deleted EIAV construct containing a p30 gene insertion would contain the p30 gene sequence in their serum.

Accordingly, various embodiments of diagnostics of the present invention comprise a serological assay for differentiating an equine inoculated with an equine infectious anemia virus (EIAV) vaccine and an EIAV infected equine. In various embodiments, the serological assay is a serological assay selected from the group consisting of an enzyme linked immunosorbant assay (ELISA), an immunodiffusion test, a fluorescent antibody test (FA), and any other test that can be used to detect antibodies in mammals. In an embodiment, the presence of antibodies to the mutated gene indicates an infected equine, whereas the absence of antibodies to the gene indicates a vaccinated equine.

In other embodiment, the diagnostic is a PCR-based diagnostic method. In various PCR based methods, embodiments detect the presence or absence of the genes, gene sequences, or fragments thereof in body fluids or tissues from a mammal and, thus, detect whether the mammal had been infected with EIAV or vaccinated. In an embodiment, the mammal is an equine. For example, and not by way of limitation, an embodiment of a PCR based diagnostic could measure the presence or absence of antibodies to a gene.

In another embodiment, a protein produced by the gene is utilized in the serological assay.

Further embodiments comprise a diagnostic kit for differentiating an equine inoculated with an equine infectious anemia virus (EIAV) vaccine and an EIAV infected equine comprising a serological assay for detecting antibodies to an accessory gene of EIAV wherein the absence of antibodies to the accessory gene indicates an inoculated equine. In an embodiment, the diagnostic kit comprises a serological assay selected from the group consisting of an enzyme linked immunosorbant assay (ELISA), an immunodiffusion test, a fluorescent antibody test (FA), and any other test that can be used to detect antibodies in mammals.

Various kits of the present invention further comprise one or more of a container, such as a Ni-NTA H is Sorb 96-well ELISA, a positive control sample, a negative control sample and an EIAV vaccine. Other components of kits of the various embodiments of the present invention will be readily apparent to those of ordinary skill in the art.

Further, it is within the scope of this invention that a diagnostic test can be used to differentiate vaccinated equines from non-vaccinated and/or infected equines by measuring the presence or absence of antibodies to the deleted gene protein.

Applicants incorporate by reference the contents, in their entirety, of U.S. Pat. No. 6,461,616, filed Sep. 9, 2000; U.S. Pat. No. 6,528,250, filed Sep. 9, 2000; U.S. Pat. No. 6,585,978, filed Sep. 9, 2000; and, U.S. Pat. No. 6,727,078, filed Apr. 27, 2000. Further Applicants incorporate by reference the contents, in its entirety, of U.S. application Ser. No. 10/627,568, field Jul. 24, 2003.

### EXAMPLES

Subcloning of S2 from EIAV_{UK}. To achieve adequate levels of recombinant S2 protein production, a series of cloned S2 gene constructs were developed and evaluated for protein expression properties. These constructs are summarized in Table 1.

The pcDNAS2 construct (Table 1) was developed to express histidine peptide-tagged S2 protein from the native EIAV S2 gene sequences. For construction of pcDNAS2 expression vector (Table 1), the EIAV S2 gene sequence was amplified from EIAV_{UK} (5) by PCR using Taq DNA polymerase (Invitrogene, Carlsbad, Calif.) with primers: FS2 (SEQ ID NO:1: GGATTATTTGGTAAAGGG) and RS2 (SEQ ID NO:2:TCATTTCTTGGTCTCTTG). The PCR products were ligated into the pcDNA4/HisMAX (Invitrogen, Carlsbad, Calif.) by TA cloning and six-histidine peptide was fused to the 5′ end of S2. This vector allows for expression of a (His)₆ tagged S2 protein from the CMV and phage T7 promoters.

Embodiments of the present invention are not limited to the above method of amplification. Suitable examples of other methods of gene amplification may be found in Wigler, et al. PNAS USA (1980) 77: 3567-3570 describes the transformation of mammalian cells with an amplifiable dominant-acting gene. Lee, et al. Nature (1981) 294: 228-232 (describing glucocorticoid regulation of expression of dihydrofolate reductase cDNA in mouse mammary tumor virus chimeric plasmids); Numberg et al. PNAS USA (1978) 75: 5553-5556; Wahl et al. J. Biol. Chem. (1979) 254: 8679-8689 (for descriptions of gene amplification); U.S. Pat. No. 4,442,203; U.S. Pat. No. 5,411,860; U.S. Pat. No. 5,445,954; U.S. Pat. No. 5,571,690; and, the like. Methods of gene amplification are common in the art and any method may be used.

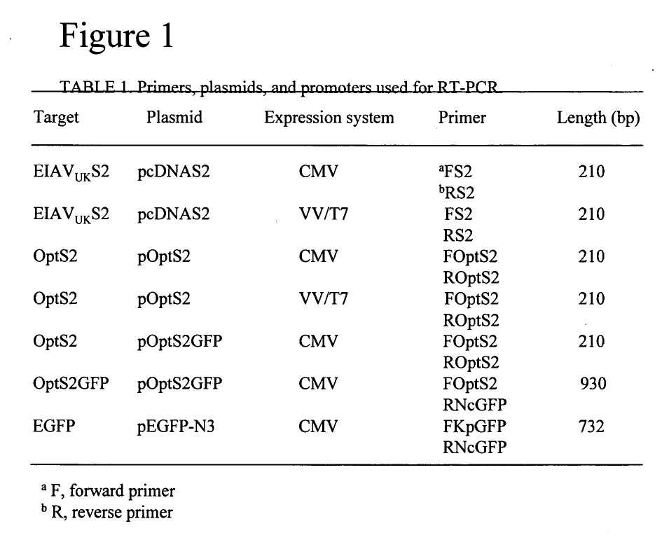

The pOptS2 construct was designed to express recombinant histidine peptide tagged S2 from an S2 gene modified for codon optimization. For construction of pOptS2 expression vector, the Codon Usage Database (www.kazusa.or.jp) was used to optimize the codons of the EIAV_{UK} S2 gene sequence for maximum expression in mammalian and E. coli cells. This codon-optimized S2 sequence (FIG. 1) was subcloned into the pcDNA3.1/mycHis vector (Invitrogene, Carlsbad, Calif.) using the Nhe I and Hind III restriction enzyme sites, and a (His)₆ peptide was fused to the 3′ end of S2. This vector allows for expression of the histidine peptide tagged S2 from the CMV and phage T7 promoters.

The pOptS2GFP construct (Table 1) was designed to express recombinant histidine peptide tagged S2 as a fusion protein with enhanced green fluorescent protein (EGFP). To construct the pOptS2GFP expression vector, the codon-optimized S2 gene sequence was amplified from the pOptS2 plasmid by PCR using the Pfu Turbo Polymerase (Strategene, La Jolla, Calif.) and primers: FoptS2 (SEQ ID NO:3: GGCCGTGCTAGCATGGGCCTGTTCGGCAAG) and RoptS2 (SEQ ID NO:4: CTAGTGGGTACCCTTCTTGGTCTCCTGCTTGCGGCG). The digested fragments were ligated into the pEGFP-N3 plasmid (Clontech, Palo Alto, Calif.), which contained the EGFP, through the Nhe I and Kpn I restriction enzyme sites. Expression from the pOptS2GFP vector was driven by the CMV promoter, and the EIAV S2—EGFP fusion protein was designated HS2G.

In addition to the expression plasmids described above for expression of mammalian cells, expression plasmids were also developed for production of recombinant proteins in E. coli. In the construction of the p80LOS2GFP expression vector for expressing the HS2G fusion protein in E. coli, the codon-optimized S2 gene sequence was amplified from the pOptS2 plasmid by PCR using primers: FS2SacI (SEQ ID NO:5: GTACCAGAGCTCATGGGCCTGTTCGGCAAG) and RoptS2 (shown above). The resulting fragments were double-digested using the Sac I and Kpn I restriction enzymes and ligated into the pQE-Trisystem plasmid (QIAGEN, Valencia, Calif.) to generate the intermediate vector pOS2QE. Next, the EGFP gene sequence was amplified from the pEGFP-N3 plasmid using the primers: FHindGFP (SEQ ID NO:6: ATGGTGAAGCTTATCGAGGGAAGGGTGAGCAAGGGCGAGGAGCTGTTC) and RxhoIG (SEQ ID NO:7:GAATTCCTCGAGCTTGTACAGCTCGTCCATGCCGAG) and subcloned into the pOS2QE plasmid using the Hind III and Xho I restriction enzyme sites. This plasmid was designated pOS2GFP. The S2-EGFP gene sequence was then excised from the pOS2GFP plasmid and ligated into the pQE-80L plasmid (QIAGEN, Valencia, Calif.) using the Sac I and Sal I restriction enzyme sites. Expression of the HS2G fusion protein from the final p80LOS2GFP expression vector was driven by the CMV promoter.

All expression vectors were verified by DNA sequencing analyses.

Transfection and protein expression in COS7 cells. Monkey kidney (COS7) cells were maintained in Dulbecco's modified Eagle's medium (DMEM) supplemented with 10% fetal bovine serum (FBS), penicillin (200 units/ml), and streptomycin (100 μg/ml) in a humidified atmosphere with 5% CO₂ at 37° C. COS7 cells were transfected with the S2 expression vectors using the PolyFect Transfection reagent according to the manufacturer's protocol (QIAGEN, Valencia, Calif.). When expression was to be driven by the T7 promoter, 80% confluent COS7 cells that had been seeded for 24 hr were infected by recombinant vaccinia virus vTF7-3 that synthesizes bacteriophage T7 RNA polymerase at MOI=30 for 30 min prior to transfection, as described elsewhere (6).

Other embodiments of the invention may use other cell lines or other productions systems, such as prokaryotic, bacterial and other eukaryotic cell lines. Further, in embodiments utilizing a promoter, the promoter may be chosen to accommodate the cell line.

Protein expression in E. coli. Previous studies in our lab indicated a high cytotoxic effect of expressed S2 protein in E. coli (unpublished data). To minimize this cytotoxic effect, the E. coli strain XL1-blue (Stratagene, La Jolla, Calif.) was utilized for S2 antigen expression. The presence of the lac I^{q} mutation efficiently blocks transcription by producing high amounts of the lac repressor. Following transformation with the p80LOS2GFP expression vector, the transcription and expression of HS2G was rapidly induced by the addition of IPTG, which bound the lac repressor proteins, thus preventing their binding with the lac O, to permit the initiation of transcription.

Following transformation with the p80LOS2GFP expression vector, the recombinant E. coli XL:p80LOS2GFP cells were pre-cultured in 5 ml Luria broth (LB) medium supplemented with 2 g/l glucose and 100 μg/ml ampicillin overnight at 37° C. with vigorous shaking. The overnight culture was used to inoculate 100 ml LB medium. The expression of HS2G protein was induced by addition of 1 mM isopropyl-belta-D-thiogalactopyranoside (IPTG) to the log-phase culture. The bacterial pellets were harvested at 4 h post induction by centrifugation at 6,000×g for 10 minutes, and stored at −80° C. until needed.

Purification of HS2G under denaturing conditions. In preliminary experiments, it was indicated that HS2G protein expressed in E. coli was localized predominantly in relatively insoluble inclusion bodies. To recover HS2G from these inclusion bodies, we employed 8 M urea to denature the proteins. In brief, bacterial pellets were lysed using buffer A (100 mM NaH₂PO₄, 10 mM Tris.HCl, and 8M urea, pH 8.0) at 5 ml per gram wet cell weight. After 45 minutes of shaking, nucleic acids and cell debris were removed by centrifugation at 10,000×g for 15 minutes. The lysates were loaded onto a 5 ml IMAC column (Amersham, Piscataway, N.J.) that was pre-equilibrated with the buffer A. The column was then washed with wash buffer (100 mM NaH₂PO₄, 10 mM Tris.HCl, 8M urea, pH 6.3), and the denatured HS2G protein was eluted using the elution buffer (100 mM NaH₂PO₄, 10 mM Tris HCl, and 8 M urea, pH 4.9). Fractions collected at OD₂₈₀ were measured for their protein content and analyzed by SDS-PAGE and Western blot assay.

Protein assay. Protein concentrations were determined using the Bio-Rad protein assay reagent and bovine gamma globulin (IgG) was used as a standard (Bio-Rad, Hercules, Calif.).

Protein Refolding. HS2G proteins purified under denaturing conditions were refolded by stepwise dialysis against buffer 1 (6 M urea, 0.5 M NaCl, 0.1 mM DTT, and 0.5 mM PMSF) at 4° C. This dialysis buffer was then diluted stepwise against buffer 2 [25 mM Tris-HCl (pH 7.5) and 150 mM NaCl] until the urea concentration decreased to 2 M over 1 day, at which time the proteins were dialyzed against buffer 3 [25 mM Tris·HCl (pH 7.5), 0.1 M NaCl, and 5% glycerol].

SDS-PAGE and Western blot analysis of recombinant S2 and HS2G proteins in cell lysates. For sodium dodecyl-polyarylamide gel electrophoresis (SDS-PAGE) analysis of purified S2 proteins, samples were solubilized in protein sample buffer (2.5% SDS, 1% p-mercaptoethanol, pH 6.8, 0.05 M Tris.HCl, 0.01% bromophoenol blue, 3 mM EDTA, and 10% glycerol) prior to being heated at 95° C. for 5 minutes. The proteins were then resolved on 4-15% polyacrylamide gel (Bio-Rad, Hercules, Calif.) and visualized by Coomassie blue R-250 staining.

For analysis of intracellular S2 protein expression by Western blot, cell lysates were first resolved on a precast 4-20% polyacrylamide gel (Bio-Rad, Hercules, Calif.) and then transferred electrophoretically to a PVDF membrane (Millipore, Billerica, Mass.) at 100 V for 1 hour. The membrane was first blocked for 1 hour with Tween-PBS (1.37 M NaCl, 27 mM KCl, 81 mM Na₂HPO₄.12H₂O, 15 mM KH₂PO₄, and 0.1% Tween 20) containing 5% powdered nonfat milk, and then incubated with primary antibodies [rabbit anti-S2 serum (1:200) or GFP monoclonal antibody (1:2000) (Promega, Madison, Wis.) at room temperature for 1 hour. Following three 5-minute washes with Tween-PBS, membranes were incubated with secondary antibodies [horseradish peroxidase (HRP)-conjugated anti-rabbit immunoglobulin G (IgG) (1:10,000) or HRP-conjugated anti-mouse IgG (1:4000) (Sigma, St. Louis, Mo.) for 1 hour and then treated to three final washes. Specific proteins were visualized with Western lightning chemiluminescence reagent plus substrate mixture (Perkin Elmer, Boston, Mass.).

Quantification of S2-specific mRNA by RT-PCR assay. To evaluate the transcription levels of the various S2 gene constructs in transfected cells, COS7 cells were transfected with the S2 expression plasmids outlined in Table 1 and harvested 48 hours post-transfection. The mRNA from transfected cells was then prepared using a one-step RT-PCR system (Pierce, Rockford, Ill.) and performed according to manufacturer instructions. Levels of β-actin mRNA were assayed separately as an internal reference. The β-actin primer sets were as follows: forward primer, SEQ ID NO:8: 5′-ATGGATGATGATATCGCCGC-3′; reverse primer, SEQ ID NO:9: 5′-GAGTCCATCACGATGCCAGT-3′. Levels of mRNA in the cell lysates were measured and quantified using Kodak 1-D gel electrophoresis analysis software.

Sources of immune sera. A panel of 65 immune serum samples was obtained from EIAV test positive field horses and from horses experimentally infected with the attenuated EIAV_{UK}ΔS2 vaccine strain (20). All field and experimental horses were shown to be seropositive in USDA reference AGID (Coggins) assays. Control horse sera were obtained from uninfected horses and confirmed as seronegative in the Coggins AGID assay. Rabbit antiserum to S2 protein was produced by immunization of rabbits with a synthetic S2 peptide preparation.

Detection of 52-specific serum antibodies by ELISA. Serum reactivity to the recombinant purified HS2G protein protein was assayed in a standard ELISA format and compared to reactivity in an affinity ELISA format. For the standard ELISA procedure, 96-well microplates (Immunolon 2, Dynatech, Laboratories, Inc., Chantilly, Va.) were coated overnight with HS2G protein diluted in 0.05 M sodium bicarbonate buffer (pH 9.6) to a final concentration of 10 μg/ml. The following day the plates were washed using PBS-Tween (0.05%), and blocked with blocking buffer (PBS containing 5% calf serum, 5% non-fat dry milk, and 0.25% Tween 20) for 1 hour at room temperature. After washing with PBS-Tween, 100 μL of 1:25 diluted horse serum in blocking buffer was applied to the plates and incubated for 30 minutes at room temperature. The plates were washed again, and incubated with 100 μl of 1:35,000 diluted anti-horse IgG HRP conjugate antibody (Sigma, St. Louis, Mo.) for 45 minutes at room temperature. Following a final PBS-Tween wash, 100 μl of the substrate 3,3′,5,5′-tetramethyl benzidine (TMB) (Sigma, St. Louis, Mo.) was added to each well, color was allowed to develop for 10 minutes before the reaction was stopped by the addition of 100 μl of 2 M H₂SO₄. The extent of the reactions was determined spectrophotometrically with a microplate reader (MR5000; Dynatech Laboratories Inc., Chantilly, Va.) at 450 nm using the software BIOLINX. In the analyses using rabbit anti-S2 serum as primary antibody in the ELISA assay, the rabbit immune serum was 1:200 diluted in blocking buffer, and anti-rabbit IgG HRP conjugate antibody diluted 1:10,000 (Sigma, St. Louis, Mo.) was used as the secondary antibody.

For the affinity ELISA procedure (NN-ELISA), HS2G protein was diluted in PBS/BSA(0.2%) buffer to a final concentration of 10 μg/ml. Ni-NTA H is Sorb plates (QIAGEN, Valencia, Calif.) were coated with 100 μl per well of diluted HS2G by gentle shaking for 1.5 hours at room temperature. Blocking was not required as the plates were pre-blocked with BSA by the manufacture. PBS was used as wash buffer, and PBS/BSA was employed to dilute primary and secondary antibodies. All subsequent procedures were as described above for the standard format.

Horse sera known to be negative or positive for EIAV antibodies were tested in parallel in each assay as controls. Values for test serum samples were derived by subtracting the adsorption values obtained at an OD₄₅₀ of the negative control serum sample. All data were representative of three independent experiments.

Production of recombinant ELAV p26. For the production of EIAV p26 capsid antigen, the p26 gene of EIAV was subcloned from EIAV_{UK}, modified with a (His)₆ tag for purification, and inserted into pQE-80L expression plasmid as described above. E. coli cells were transformed with the p26 expression plasmid and precultured in 5 ml Luria broth (LB) medium supplemented with 2 g/l glucose and 100 μg/ml ampicillin overnight at 37° C. with vigorous shaking. The overnight culture was used to inoculate 100 ml LB medium. The expression of p26 protein was induced by addition of 1 mM isopropyl-belta-D-thiogalactopyranoside (IPTG) to the log-phase culture. The bacterial pellets were harvested at 4 h post induction by centrifugation at 6,000×g for 10 minutes. The cells were lysed with a bacterial protein extraction reagent (Pierce, Rockford, Ill.), filtered through 0.45 μm filters, and applied to a column equilibrated with the binding buffer [10 mM Na₂HPO₄ 2H₂O, 10 mM NaH₂PO₄.H₂O, and 0.5 M NaCl (pH7.4)] according to the manufacturers directions (Amersham, Piscataway, N.J.). The column was then washed with the wash buffer containing [10 mM Na₂HPO₄.2H₂O, 10 mM NaH₂PO₄.H₂O, 0.5 M NaCl, and 10 mM imidazole (pH7.4)] at a flow rate of 5 ml/min. The HS2G protein was eluted using the elution buffer [10 mM Na₂HPO₄.2H₂O, 10 mM NaH₂PO₄.H₂O, 0.5 M NaCl, and 250 mM imidazole (pH7.4)]. To examine the quality of the purified p26 protein, fractions collected at OD₂₈₀ were measured for their protein content and analyzed by SDS-PAGE and Western blot assays.

Detection of serum antibodies to p26. For p26-specific NN-ELISA procedures, purified recombinant p26 antigen was dissolved in PBS/BSA (0.2%) to a final concentration of 5 μg/ml and horse serum diluted to 1:200 in PBS/BSA. The other procedures for the p26 assay were as described above for NN-ELISA to HS2G.

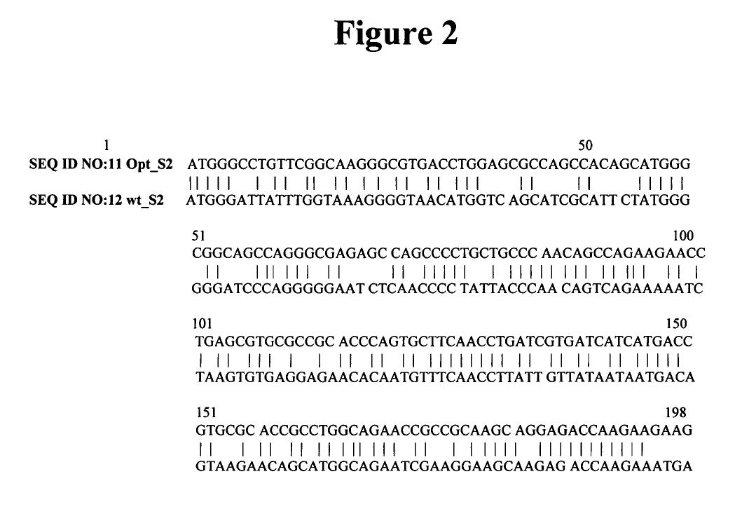

Expression of recombinant S2 antigen in mammalian cells. To achieve reproducible and adequate expression of a recombinant S2 antigen, a series of S2 gene expression constructs was developed and evaluated. Comparative transcription and protein expression levels from this series of antigen expression constructs are summarized in FIG. 2. Our first attempts to express the EIAV S2 protein began with the pcDNAS2 expression plasmid containing the EIAV_{UK} S2 sequence. This plasmid was transfected into the mammalian cell line COS7, and mRNA expression (FIG. 2A) and recombinant S2 protein production (FIG. 2B) were evaluated by RT-PCR and Western blot analyses, respectively. These assays indicated a lack of detectable S2 protein expression in the transfected cells, despite the presence of detectable S2 mRNA from both the T7 and CMV promoters.

The transcription and translation efficiency of the pcDNAS2 expression plasmid was next evaluated as a possible reason for lack of expression. It was determined that the equine S2 arginine codons AGG and AGA are less frequently utilized in COS and E. coli cells, probably due to a lower abundance of corresponding tRNAs. Accordingly, we constructed the pOptS2 expression plasmid using the optimized EIAV_{UK} S2 (OptS2) codon sequence (FIG. 1), and following transfection of COS7 cells, S2 mRNA and protein expression was evaluated using both the T7 and CMV promoters (FIG. 2). The results of these assays indicated that the codon optimization enhanced S2 mRNA expression by the T7 or CMV promoters by 3.2-fold compared to the parental S2 gene, but that this increased mRNA expression still failed to yield detectable S2 protein in the transfected cells.

These observations suggested a potential instability of the expressed S2 protein in transfected cells. Small peptides are often degraded in vivo, and as S2 has a molecular weight of only 7.2 kDa (27), degradation was considered a possible reason for the lack of S2 expression in vitro. To stabilize and protect our expressed protein from degradation, a fusion protein was constructed by attaching the EGFP protein to the carboxyl terminus of the S2 protein (Material and Methods). This fusion protein was designated as HS2G, and the plasmid expressing this fusion protein was termed pOptS2GFP. To assess the efficacy of this expression construct, COS7 cells were transfected as described above and assayed for mRNA and recombinant protein expression. The data in FIG. 2 demonstrates substantial mRNA production in the transfected cells, and for the first time, detectable levels of the recombinant S2 protein as part of the HS2G fusion protein by immunoblotting with the rabbit anti-S2 serum. The expression of the fusion protein was also verified using mouse anti-EGFP as a primary antibody (data not shown). These results indicate that the HS2G fusion protein is successfully recognized by anti-S2 antibodies and anti-EGFP. All combined, these data suggest that construction of the HS2G fusion protein successfully prevented degradation of the S2 protein in COS7 cells.

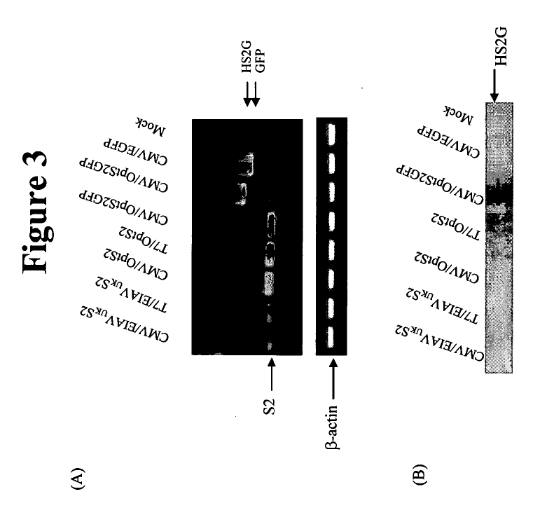

Production and purification of S2 antigen from E. coli. In parallel to our development of an S2 antigen expression system in the mammalian COS7 cells as a basis for investigating S2 function in viral replication, we also examined the expression levels of the various recombinant antigen constructs in E. coli to provide high yield production system for a potential diagnostic antigen. For these purposes, the various engineered S2 genes were cloned into the prokaryotic expression plasmid pQE-80L, and their expression levels evaluated in transformed E. coli. As observed with expression in COS7 cells, we observed in preliminary experiments that there was no detectable S2 antigen produced in E. coli cells transformed with either the parental S2 gene or the codon optimized S2 gene, while substantial levels of the recombinant HS2G fusion protein were detected in E. coli transformed with the p80LOS2GFP expression plasmid (data not shown). FIG. 3 summarizes representative data on HS2G expression in transformed E. coli cells. As described in the Materials and Methods, p80LOS2GFP plasmid was transformed into E. coli XL cells. The expression of HS2G was verified by flow cytometric analysis as shown in FIG. 3A. Approximately 60% recombinant cells expressed HS2G protein at 4 hr post induction with IPTG. This experimental study also demonstrated that the optimal time frame for cell harvest to be four hours post IPTG-induction because it presented the highest expression level of the fusion protein over the post induction period 0 to 5.5 hr.

To evaluate the possible methods for extracting the HS2G protein from the transformed bacteria, soluble proteins were extracted from a transformed E. coli cell pellet by lysis using the commercially available bacterial protein extraction reagent, filtered and evaluated by SDS-PAGE (Lane 1, FIG. 3B) and Western blot (Lane 4, FIG. 3C), respectively. To extract insoluble proteins from the transformed cells, an identical E. coli cell pellet was lysed using a 8M urea (Material and Methods) and the extracted proteins evaluated by both SDS-PAGE (Lane 2, FIG. 3B) and Western blot (Lane 5, FIG. 3C). The data in FIGS. 3B and 3C indicates that the HS2G protein expressed in transformed E. coli was predominantly in the insoluble fraction of the cell pellet, thus requiring 8M urea for extraction. The extracted HS2G protein was then purified from the total cellular extract by affinity chromatography on nickel-charged IMAC column according to the manufacturer's procedures. This purification procedure yielded a total of about 4.5 mg of HS2G protein from 500 ml culture broth without detectable contaminating proteins (Lane 3, FIG. 3B and Lane 6, FIG. 3C). To address the concern that denatured HS2G protein may be less reactive with serum antibodies and thus a less useful as antigen in an ELISA assay, the affinity column purified denatured HS2G protein was refolded by stepwise dialysis, as described in Materials and Methods. Typically, the final refolded HS2G protein yield was about 50% of the starting protein antigen recovered from the affinity column.

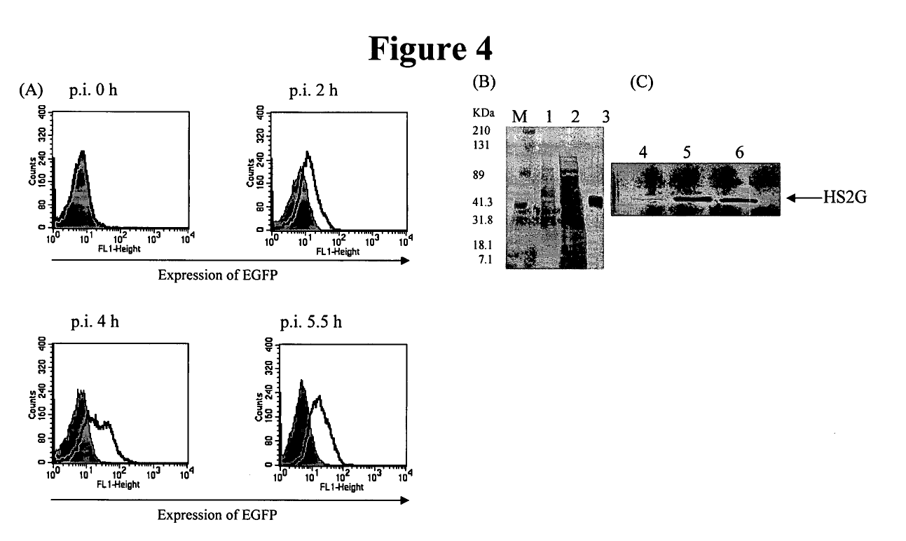

Development and optimization of an HS2G-based ELISA. Having achieved the necessary goal of S2 antigen production, we next sought to evaluate the utility of an S2-based serological assay to detect and discriminate EIAV-infected horses from horses inoculated with attenuated vaccine virus lacking S2 expression. To address this issue, the reactivity of the purified HS2G protein was initially examined in a standard ELISA for reactivity with a reference rabbit immune serum and with a reference EIAV positive horse serum. The data in FIG. 4A demonstrates that HS2G protein displayed significant reactivity to the rabbit anti-S2 polyclonal antibodies, but relatively low level reactivity was observed with the reference horse serum. The relatively low level of S2-specific antibody reactivity was consistent with previous reports of low antibody titers to S2-specific synthetic peptides in ELISA formats.

To increase the sensitivity of the HS2G in vitro ELISA test system, we modified the standard ELISA by employing Ni-NTA H is Sorb 96-well ELISA plates to affinity adsorb the HS2G protein antigen through the (His)₆ tag of the fusion protein. Thus, HS2G protein was coated onto the Ni-NTA plates, and the reactivity of the adsorbed antigen with the reference rabbit and horse immune sera was evaluated. As shown in FIG. 4B, a similar level of reactivity of HS2G to rabbit anti-S2 antibodies was achieved in NN-ELISA as compared to the standard ELISA above. In contrast, however, the reactivity of the horse immune serum in the NN-ELISA was increased by about 3-fold compared to the standard ELISA format, while the background reactivity with the negative control horse serum was reduced by about 2-fold in the NN-ELISA compared to the standard ELISA procedure. These data indicated the potential of the NN-ELISA as a format for further development and assessment.

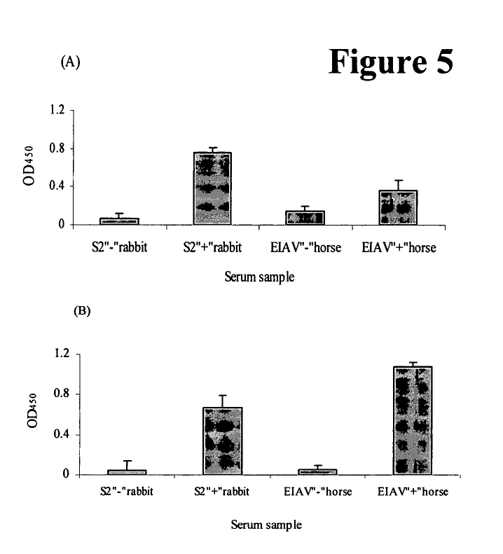

To evaluate in more detail the sensitivity and specificity of HS2G protein and to validate the application of the NN-ELISA test system for identifying EIAV-infected horses, we appraised the reactivity of a coded panel of reference sera from EIAV negative horses, EIAV-infected horses, and EIAV_{UK}ΔS2 vaccinated horses sera to HS2G in the NN-ELISA test system, and compared these results to those obtained from the standard ELISA test system (FIG. 5). The results indicated that the NN-ELISA was superior to the standard ELISA procedure both in the sensitivity and specificity of serum reactivity. For example, all five of the control sera from uninfected horses were seronegative in the NN-ELISA, while one of the uninfected horse serum samples displayed reactivity in the standard ELISA format. Similarly, all eight of the immune serum samples from EIAV_{UK}ΔS2 vaccinated horses were seronegative in the NN-ELISA format, while two of these vaccinate sera displayed low levels of seroreactivity in the standard ELISA. Taken together, these observations demonstrate the absence of nonspecific seroreactivity to the HS2G antigen in the context of the NN-ELISA format, providing the specificity required to avoid false indications of EIAV-infected horses. In addition to the superior specificity, the NN-ELISA also yielded substantially higher levels of reactivity of all six reference immune sera from EIAV-infected horses compared to the standard ELISA. The seroreactivity levels achieved in the NN-ELISA format for the reference immune serum samples ranged from 3 to 20-fold higher than the respective serum reactivity realized in the standard ELISA. Importantly, for three of the EIAV positive immune sera (horses 561, 562, and 95), the NN-ELISA provides a strongly positive signal, where the standard ELISA reactivity borders on a negative level of reactivity. Thus, these data demonstrate the preferred sensitivity and specificity of the NN-ELISA for accurately detecting EIAV-infected horses with relatively low levels of S2-specific serum antibodies, while reliably distinguishing uninfected or vaccinated horses lacking S2-specific serum antibodies.

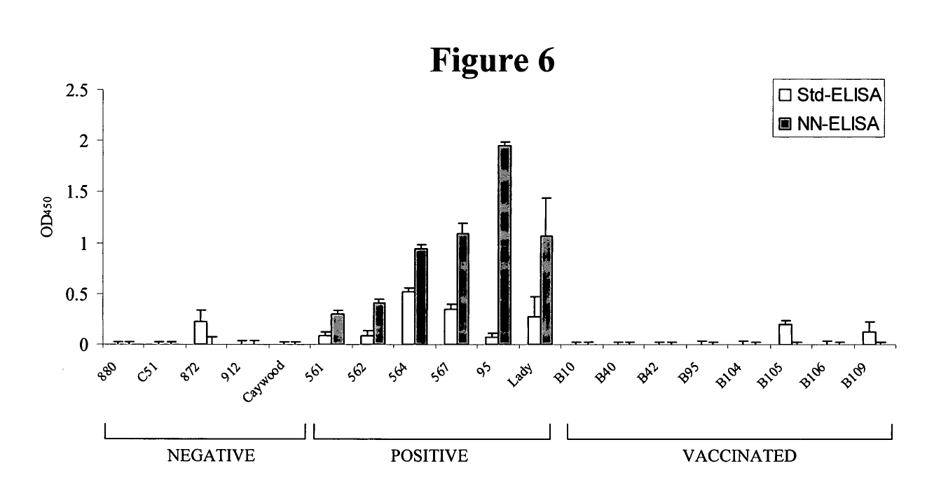

Application of NN-ELISA as a diagnostic assay for ELAV infection. Having identified the NN-ELISA as the superior format as the basis for the S2 serological diagnostic assay, we next sought to compare the sensitivity and specificity of this assay with an ELISA detecting serum antibodies to the EIAV capsid protein p26. For these comparisons, we assembled a coded panel of reference serum from 7 uninfected horses, 18 field infected horses, and 21 EIAV_{UK}ΔS2 vaccinated horses (FIG. 6). All of the field infected horses and vaccinates were confirmed as seropositive in the Vira-CHEK p26 ELISA test (Synbiotics Corp.), while all of the uninfected horses were seronegative in this diagnostic assay (data not shown). The p26 serum antibody reactivity observed in our ELISA assay (FIG. 6) was completely in agreement with the Vira-CHEK assay in identifying virus infected and uninfected horses, thus validating our p26 ELISA for comparison to the NN-ELISA diagnostic assay.

The NN-ELISA seroreactivity data in FIG. 6 demonstrated a high level of sensitivity and specificity for identifying and distinguishing EIAV-infected horses from uninfected horses and EIAV_{UK}ΔS2-vaccinated horses. All 7 of the uninfected horse sera were found to be unreactive in the NN-ELISA format. Similarly, all 21 of the immune serum samples from the vaccinated horses failed to display any detectable reactivity in the NN-ELISA, confirming the lack of S2-specific antibodies in these horses. In contrast to these seronegative sera, the NN-ELISA identified 16 of 18 EIAV-infected horses as seropositive, with two infected horse sera (#11 and #13) failing to display serum antibody reactivity to the HS2G antigen.

Combining the NN-ELISA serological data presented in FIGS. 5 and 6, it can be calculated that the NN-ELISA was 100% accurate in identifying S2 antibody negative serum from uninfected horses (12/12); there were no detectable false positives. The NN-ELISA achieved 92% sensitivity in identifying EIAV-infected horses (22/24) by S2 serum antibody reactivity. While the current studies utilize a relatively limited number of test samples, the data clearly indicate the sensitivity and specificity of the NN-ELISA assay as a potential diagnostic assay for detecting EIAV-infected horses and distinguishing them from horses inoculated with attenuated EIAV vaccine strains lacking S2 gene expression.

It should be noted here that the diagnostic serological assays to detect serum antibodies to S2 may also be used to distinguish EIAV-infected horses from horses immunized with other EIAV vaccine formats, including inactivated viral particles, envelope or core protein subunit vaccines, or DNA or live vector vaccines expressing viral envelope or core proteins. None of these immunization regimens are expected to elicit host antibody responses to the S2 protein, thus providing a serological means of distinguishing infected from vaccinated horses. To increase the confidence levels of an S2 serological assay, it is possible to combine the detection of S2-specific antibodies with a complementary detection of specific antibodies to appropriate EIAV virion proteins (e.g., p26) for infected horses and detection of antibodies to an antigenic marker incorporated in the EIAV vaccine.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims. Further, all patents and other publications mentioned herein are herby incorporated by reference.

1. A diagnostic method for differentiating an equine inoculated with an equine infectious anemia virus (EIAV) vaccine, wherein the vaccine comprises a gene mutated EIAV, and an EIAV infected equine comprising a serological assay for detecting antibodies to the gene.

2. The diagnostic of claim 1, wherein absence of antibodies to the gene indicates an inoculated equine.

3. The diagnostic method of claim 1, wherein the serological assay is a serological assay selected from the group consisting of an enzyme linked immunosorbant assay (ELISA), an immunodiffusion test, a fluorescent antibody test (FA), and any other test that can be used to detect antibodies in mammals.

4. The diagnostic method of claim 1 wherein the serological assay detects the presence or absence of a gene, gene sequence, or fragment thereof.

5. The diagnostic method of claim 1, wherein the protein produced by the gene is utilized in the serological assay.

6. A diagnostic kit differentiating an equine inoculated with an equine infectious anemia virus (EIAV) vaccine, wherein the vaccine comprises a gene mutated EIAV, and an EIAV infected equine comprising a serological assay for detecting antibodies to the gene wherein the absence of antibodies to the gene indicates an inoculated equine.

7. A diagnostic method for differentiating an equine inoculated with a gene mutated equine infectious anemia virus (EIAV) marker vaccine and an EIAV infected equine comprising a serological assay for detecting antibodies to the gene wherein the presence of antibodies to the gene indicates an inoculated equine.


SEQUENCE LISTING (12 sequences)

SEQ ID NO: 1 - DNA, 18 bp, Artificial
  pimer
ggattatttggtaaaggg

SEQ ID NO: 2 - DNA, 18 bp, Artificial
  primer
tcatttcttggtctcttg

SEQ ID NO: 3 - DNA, 30 bp, Artificial
  primer
ggccgtgctagcatgggcctgttcggcaag

SEQ ID NO: 4 - DNA, 36 bp, Artificial
  primer
ctagtgggtacccttcttggtctcctgcttgcggcg

SEQ ID NO: 5 - DNA, 30 bp, Artificial
  primer
gtaccagagctcatgggcctgttcggcaag

SEQ ID NO: 6 - DNA, 48 bp, Artificial
  primer
atggtgaagcttatcgagggaagggtgagcaagggcgaggagctgttc

SEQ ID NO: 7 - DNA, 36 bp, Artificial
  primer
gaattcctcgagcttgtacagctcgtccatgccgag

SEQ ID NO: 8 - DNA, 20 bp, Artificial
  primer
atggatgatgatatcgccgc

SEQ ID NO: 9 - DNA, 20 bp, Artificial
  primer
cagtccatcacgatgccagt

SEQ ID NO: 10 - protein, 8 aa, Equine infectious anemia virus
RRKQETKK

SEQ ID NO: 11 - DNA, 198 bp, Equine infectious anemia virus
atgggcctgttcggcaagggcgtgacctggagcgccagccacagcatgggcggcagccagggcgagagccagcccctgctgcccaacagccagaagaacctgagcgtgcgccgcacccagtgcttcaacctgatcgtgatcatcatgaccgtgcgcaccgcctggcagaaccgccgcaagcaggagaccaagaagaag

SEQ ID NO: 12 - DNA, 198 bp, Equine infectious anemia virus
atgggattatttggtaaaggggtaacatggtcagcatcgcattctatggggggatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatga